(19) United States
(12) Patent Application Publication
(10) Pub. No.: US 2026/0188520 A1
(43) Pub. Date: Jul. 2, 2026

(54) SIMULATION BASED PROPHYLAXIS AGAINST MUSCULOSKELETAL DAMAGE

(71) Applicant: International Business Machines Corporation, Armonk, NY (US)

(72) Inventors: Sarbajit Kumar Rakshit, Kolkata (IN); Sudheesh S. Kairali, Kozhikode (IN); Binoy Thomas, Kozhikode (IN)

(21) Appl. No.: 19/007,649

(22) Filed: Jan. 2, 2025

(51) Int. Cl. G16H 50/50; G06Q 10/0633; G16H 10/60; G16H 40/67

(52) U.S. Cl. CPC G16H 50/50; G06Q 10/0633; G16H 10/60; G16H 40/67

(57) ABSTRACT

A computer-implemented method for providing a prophylaxis against injuries by identifying a set of tasks within a workflow. The computer-implemented method includes simulating performance of the tasks using a skeletal model within a computer simulation, the skeletal model being defined by joints and articulations of the joints, aggregating stresses at the joints over time into an aggregate stress and an aggregate load, comparing the stresses to a recommended stress limit and a recommended load limit, and recommending a reconfiguration of the set of tasks in response to one of the stresses exceeding the recommended load limits.

### BACKGROUND

The present invention generally relates to prevention of musculoskeletal problems, and more specifically, to a simulation based prophylaxis against musculoskeletal damage.

Performance of repetitive activities without variation can cause a buildup of joint damage in the utilized joints. Without sufficient rest periods for a body to repair the naturally occurring damage from the repetitive activities long term and/or permanent musculoskeletal problems can develop.

### SUMMARY

Embodiments of the present invention are directed to a computer-implemented method for protecting against musculoskeletal damage. A non-limiting example of the computer-implemented method includes providing a prophylaxis against injuries by identifying a set of tasks within a workflow. The computer-implemented method includes simulating performance of the tasks using a skeletal model within a computer simulation, the skeletal model being defined by joints and articulations of the joints, aggregating stresses at the joints over time into an aggregate stress and an aggregate load, comparing the stresses to a recommended stress limit and a recommended load limit, and recommending a reconfiguration of the set of tasks in response to one of the stresses exceeding the recommended load limits.

Embodiments of the present invention are directed to a system and a computer program product for the same.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a cloud computing environment for implementing at least some portions of the processes described herein.

FIG. 2 depicts representation of a human skeletal structure, as is stored in the simulation based prophylaxis against musculoskeletal damage;

FIG. 3 depicts a process for performing the workplace analysis;

FIG. 4 illustrates a set of joint stresses in a task sequence;

FIG. 5 illustrates a combined joint stress resulting from the task sequence.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Computing environment 100 contains an example of an environment for the execution of at least some of the computer code involved in performing the inventive methods, such as a simulation based prophylaxis against musculoskeletal damage process as shown at block 150. In addition to block 150, computing environment 100 includes, for example, computer 101, wide area network (WAN) 102, end user device (EUD) 103, remote server 104, public Cloud 105, and private Cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI), device set 123, storage 124, and Internet of Things (IoT) sensor set 125), and network module 115. Remote server 104 includes remote database 132. Public Cloud 105 includes gateway 130, Cloud orchestration module 131, host physical machine set 142, virtual machine set 143, and container set 144.

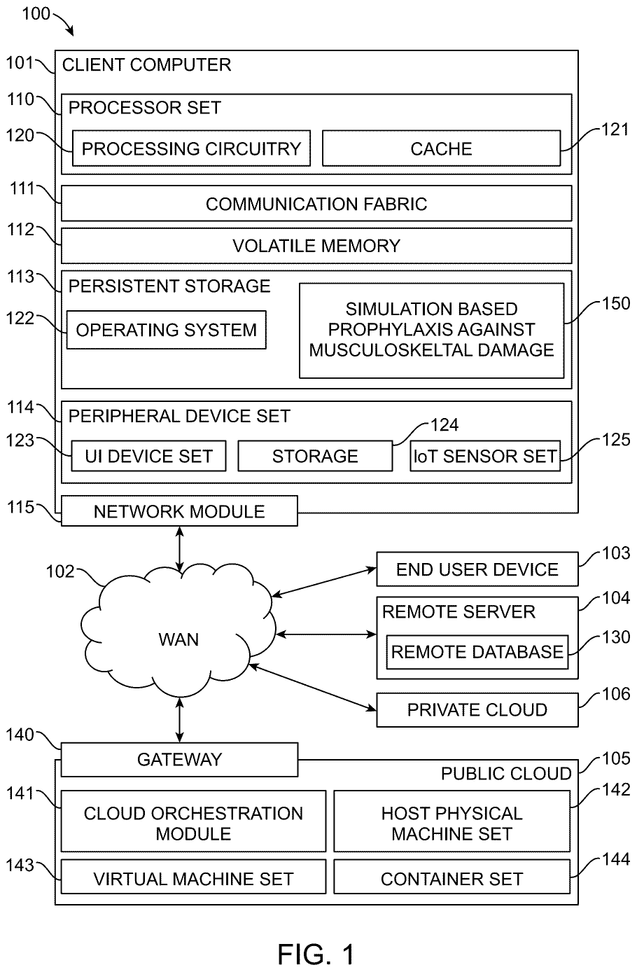

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 132. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a Cloud, even though it is not shown in a Cloud in FIG. 1. On the other hand, computer 101 is not required to be in a Cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collects and store helpful and useful data for use by other computers, such as computer 101. For example, in a hypothetical case where computer 101 is designed and programmed to provide a recommendation based on historical data, then this historical data may be provided to computer 101 from remote database 132 of remote server 104.

PUBLIC CLOUD 105 is any computer system available for use by multiple entities that provides on-demand availability of computer system resources and/or other computer capabilities, especially data storage (Cloud storage) and computing power, without direct active management by the user. Cloud computing typically leverages sharing of resources to achieve coherence and economies of scale. The direct and active management of the computing resources of public Cloud 105 is performed by the computer hardware and/or software of Cloud orchestration module 141. The computing resources provided by public Cloud 105 are typically implemented by virtual computing environments that run on various computers making up the computers of host physical machine set 142, which is the universe of physical computers in and/or available to public Cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public Cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public Cloud 105, except that the computing resources are only available for use by a single enterprise. While private Cloud 106 is depicted as being in communication with WAN 102, in other embodiments a private Cloud may be disconnected from the internet entirely and only accessible through a local/private network. A hybrid Cloud is a composition of multiple Clouds of different types (for example, private, community or public Cloud types), often respectively implemented by different vendors. Each of the multiple Clouds remains a separate and discrete entity, but the larger hybrid Cloud architecture is bound together by standardized or proprietary technology that enables orchestration, management, and/or data/application portability between the multiple constituent Clouds. In this embodiment, public Cloud 105 and private Cloud 106 are both part of a larger hybrid Cloud.

One or more embodiments described herein can utilize machine learning techniques to perform prediction and or classification tasks, for example. In one or more embodiments, machine learning functionality can be implemented using an artificial neural network (ANN) having the capability to be trained to perform a function. In machine learning and cognitive science, ANNs are a family of statistical learning models inspired by the biological neural networks of animals, and in particular the brain. ANNs can be used to estimate or approximate systems and functions that depend on a large number of inputs. Convolutional neural networks (CNN) are a class of deep, feed-forward ANNs that are particularly useful at tasks such as, but not limited to analyzing visual imagery and natural language processing (NLP). Recurrent neural networks (RNN) are another class of deep, feed-forward ANNs and are particularly useful at tasks such as, but not limited to, unsegmented connected handwriting recognition and speech recognition. Other types of neural networks are also known and can be used in accordance with one or more embodiments described herein.

ANNs can be embodied as so-called “neuromorphic” systems of interconnected processor elements that act as simulated “neurons” and exchange “messages” between each other in the form of electronic signals. Similar to the so-called “plasticity” of synaptic neurotransmitter connections that carry messages between biological neurons, the connections in ANNs that carry electronic messages between simulated neurons are provided with numeric weights that correspond to the strength or weakness of a given connection. The weights can be adjusted and tuned based on experience, making ANNs adaptive to inputs and capable of learning. For example, an ANN for handwriting recognition is defined by a set of input neurons that can be activated by the pixels of an input image. After being weighted and transformed by a function determined by the network's designer, the activation of these input neurons are then passed to other downstream neurons, which are often referred to as “hidden” neurons. This process is repeated until an output neuron is activated. The activated output neuron determines which character was input.

A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Turning now to an overview of areas that are more specifically relevant to aspects of the invention, different types of musculoskeletal problems can arise within a workforce where the workers perform repetitive activities in the industrial floor. By way of example, when one worker performs same types of movement in a repetitive manner (e.g. placing and turning a screw using a wrist rotation) without variation and for extended periods of time joint problems may eventually arrive in the worker's wrist. By way of example, lower back pain may arise due to incorrect lifting techniques and poor posture can contribute to chronic lower back pain. Similarly, neck and shoulder pain can result from awkward positions and repetitive movements can lead to neck and shoulder discomfort.

Carpal tunnel syndrome results from repetitive hand and wrist movements in awkward positions. Overuse of certain muscles due to improper movement can result in tendinitis. Incorrect lifting and movement can lead to muscle strains and tears. Poor lifting techniques can contribute to herniated discs and associated nerve pain. Repeated incorrect shoulder movements can lead to rotator cuff injuries. Prolonged standing or improper bending can result in knee pain and discomfort. Incorrect movement patterns can lead to joint pain, particularly in hips, knees, and shoulders. Awkward movements can cause ligament sprains, especially in ankles and wrists. Poor posture and movement can lead to nerve compression issues such as sciatica. Repetitive improper movements over time can contribute to degenerative joint and muscle conditions. Continuous improper movement can lead to muscle fatigue and reduced productivity. Repetitive and improper activities can cause RSIs, leading to pain and discomfort.

On any given industrial floor, workers and technology, such as robots, collaborate by engaging in tasks to accomplish an end production requirement. The tasks are frequently manual and repetitive in nature, with little variation or recovery time. This repetition and lack of recovery time for any given joint can result in one or more of the above described musculoskeletal problems. Existing solutions focus on reducing repetition of movements without further consideration for the underlying causes and cumulative effects of multiple tasks.

Turning now to an overview of the aspects of the invention, one or more embodiments of the invention address the above-described shortcomings of the prior art by employing a human skeleton model for mobility simulations of various activities while designing a workplace. The activities include both repetitive and non-repetitive activities. The process analyses recorded skeleton movements of each activity to identify joint movement patterns, frequencies, and loads on the joints resulting from the activities. Comparing these patterns with medical recommendations, the proposed system prevents musculoskeletal issues by determining an aggregate strain across multiple tasks and provides a corresponding recommendation to reduce the resultant strains and allow for proper recovery.

In some examples, the proposed system customizes repetitive movement of bone joint limits for individual workers based on health parameters. The process further assesses activities, weightlifting, and movement types that the person is expected to engage in and estimates load variations on bone joints of those activities. A simulations determines optimal activity distribution, aiding worker mobility while controlling repetitive joint movement. The process further simulates worker activities and distributes tasks among multiple workers in a manner that maintains worker efficiency and reduces any specific bone and/or joint strains. In some examples, an augmented reality system is employed to assist workers with appropriate sequences and positions to reduce strains resulting from individual tasks and, in some examples, provides the worker with techniques for converting movement into beneficial exercise

The above-described aspects of the invention address the shortcomings of the prior art by providing a recommended activity distribution, and in some examples directly implementing the recommended activity distribution. The recommended activity distribution considers the aggregate impact of all repeated tasks, whether tasks are identical, similar, or seemingly unrelated. The aggregate impact is determined using human skeleton based simulations based on analyzing recorded skeletal movements on different joints and identifying movement patterns, frequencies and loads on the different joints resulting from each type of activity.

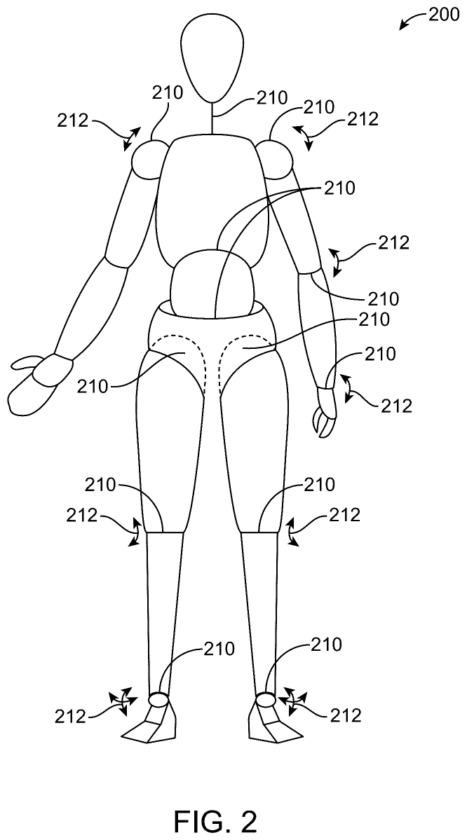

Turning now to a more detailed description of aspects of the present invention, FIG. 2 is a skeletal doll 200 representation of a human skeletal structure, as is stored in the simulation based prophylaxis against musculoskeletal damage process 150 of the computer system 100 of FIG. 1. In the interest of clarity, the illustration of FIG. 2 provides callout locations for individual features at a limited number of locations, despite those features appearing several additional times within the skeletal doll 200 defines joints 210 and articulations 212 for each joint 210.

The simulation based prophylaxis described herein is a process employs a human skeleton model (skeletal doll 200) during workplace design. The simulation uses the skeletal doll 200 for mobility simulations during various activities within the workplace, including both repetitive and non-repetitive activities. The process analyses recorded skeleton movements of the worker engaging in the activities to identify joint movement patterns, frequencies, and loads on the joints resulting from the activities. These patterns are compared with medical recommendations. The simulation prevents musculoskeletal issues by identifying cumulative loads and stresses resulting from repetition as well as from engaging in similar activities.

The proposed system analyzes repetitive movement of the bone joints relative to customized movement limits for individual workers based on health parameters of the individual workers. The process assesses activities, weight lifted, and movement types and estimates load variations on joints resulting from the same. Simulations using the skeletal doll 200 determine optimal activity distribution when designing or reconfiguring a workplace and a workflow. This aids the individual worker's mobility within a work environment while controlling repetitive joint movement. In some implementations, augmented reality systems can be implemented to assist workers with appropriate sequences and positions thereby improving their ability to perform the activities without cause damage. In some cases, the augmented reality can further assist by converting movements for workplace tasks into beneficial exercise.

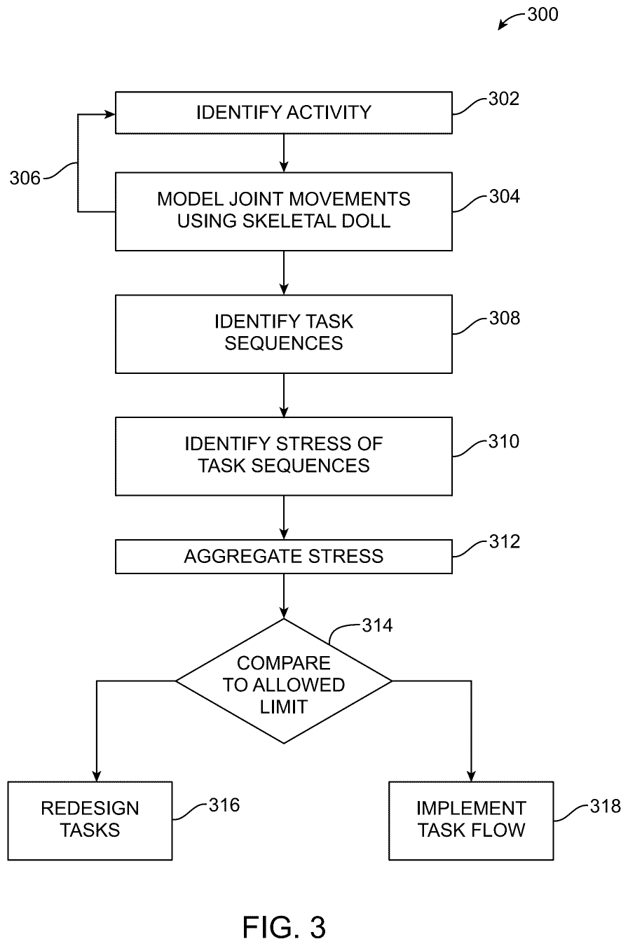

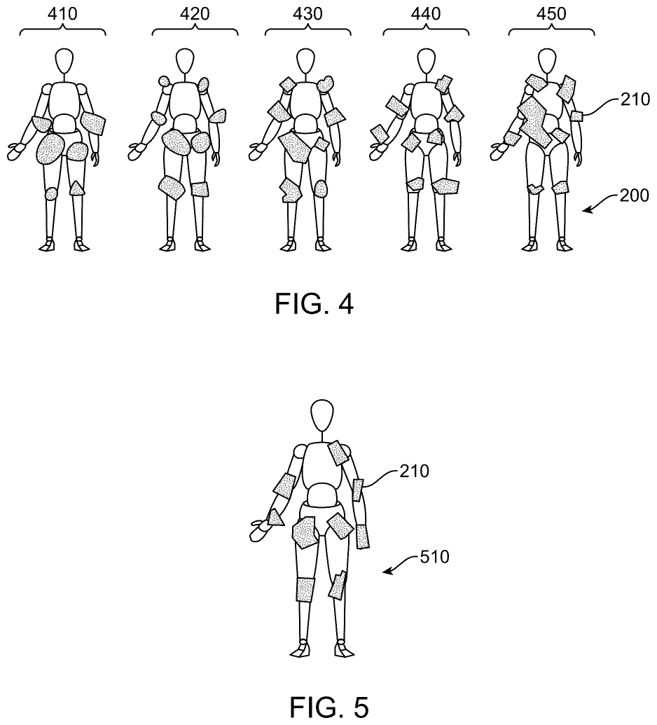

With continued reference to FIGS. 1 and 2, FIG. 3 illustrates a process 300 for performing the workplace analysis, FIG. 4 illustrates a set of joint stresses in a task sequence, and FIG. 5 illustrates a combined joint stress resulting from the task sequence.

Initially, the process 300 identifies an activity within a workplace in an identify activity step 302. Once identified, the process 300 uses the skeletal doll 200 to model identifying joint movement patterns that occur during the activity in a model joint movements step 304. The modeling includes considerations of biomechanical principles and motion analysis of workers performing the activity to identify joint movement patterns.

Within the modeling, the skeletal doll 200 can be either a two dimensional model or a three dimensional model, provided the skeletal doll 200 has the required types of structures and joint connections. Using the skeletal doll 200, the step 304 identifies and labels the joints 210, the corresponding degrees of freedom of those joints 210, as well as the types of movement (flexion/extension, abduction, adduction, rotation) that those joints are capable of engaging in.

The motion capture portion of the modeling records movements of actual humans perform the task and maps the movements of the actual humans to the joints 210 of the skeletal doll 200. In some examples, the motion capture includes a variety of body types (e.g., tall/short, heavy weight/light weight, etc.) performing the task. The model analyzes the joint angle data from the motion to identify and quantify joint movements during the activity. This quantification includes calculating ranges of motion, peak angles, and patterns of movement for each joint during the activity.

In some examples, the model considers historically captured different types of videos on how various humans have differently performed the activity and the corresponding movement, thereby allowing the model to associate variations in the movements used to accomplish a given activity with corresponding variations like size, height, reach, etc. In some examples, the capturing breaks down the recorded motion data into segments corresponding to different phases of the activity (e.g., initial stance, midstance, terminal stance in walking). The model then identifies and labels sub activities within each activity, allowing the model to define each activity as a set of sub-activities and compare the similar sub activities across different activities in order to identify similar stresses. In some examples, the modeling identifies movement patterns that change based on variations in sped, load, or other factors that impact performance of the activity.

Once the identified activity has been fully modeled within the simulation, the process 300 returns to the identify activity step 302 and identifies another activity that occurs within the workplace through a loop 306. The steps 302, 304 are looped using the loop 306 until all activities have been identified and modeled.

In some examples, modeling activities includes classifying different types of activities into categories based on common characteristics, thereby simplifying the analysis. Classifying the activities includes consideration of what joint action is being performed, such as flexion, extension, abduction, adduction, and rotation, and classifying activities using similar joint actions within the same classification. In some examples, the classifications include action types like lifting, carrying, reaching, bending, twisting, pushing, pulling, etc.

The process 300 then analyzes the recorded data to understand the specific joint movements that are consistently repeated within each activity category and frequencies of joint activities. The skeletal doll 200 then performs the activities within the simulation and the process 300 identifies the frequency (how often) and duration (how long) of the specific repetitive joint movements within each activity.

After identifying and modeling each activity, the process 300 proceeds to identify sequences of tasks that occur in a particular workplace in an identify sequences of tasks step 308.

The sequences of steps identified at step 308 includes the types of physical activities that human workers perform on the workplace floor being designed or reconfigured and includes multiple sequential activities that occur serially in order to accomplish a larger task. The analysis uses internet of things (Io)T and camera data, and workflow information to determine specific sequences of tasks that are performed including identifying activities that are performed by human workers, as well as connection activities that transition from one identified activity to another including walking, lifting, carrying, pushing, pulling, reaching, bending, and the like. Based on the identified activities within the sequence, and historically performed the activities, the process 300 use the skeleton doll 200 of the worker to simulate how the human worker performs the sequence of activities as a structured sequence, rather than as individual activities. In some examples, the simulation is worker specific and incorporates dimensional and body type information of a specific worker. By way of example, this information can include height, weight, reach, age, and/or any similar demographic details.

Based on medical data, and ergonomic data the process identifies the ergonomic impact of each activity within the sequence and assess whether the movements and joint actions align with recommended ergonomic guidelines to prevent strain and injuries. Identification of the sequences further includes identifying the duration and frequency of each activity in the sequence and considers how often certain activities are performed and how long workers are engaged in specific movements. As part of this analysis, the process uses the previously determined models to aggregate how many reparative bone movements can happen on the skeletal doll 200, and calculates the forces exerted on the body during these activities to evaluate potential strain.

The stresses associated with each task in a task sequence is illustrated graphically in FIG. 4, with tasks 410, 420, 430, 440, 450 illustrating the respective stresses of sequentially performed tasks 410, 420, 430, 440, 450 within a sequence visually as blurs on joints 210 of skeletal doll 200 with the size of the blur indicating the magnitude of stresses occurring within the task 410, 420, 430, 440, 450.

The stresses associated with each task 410, 420, 430, 440, 450 in the sequence of tasks are aggregated into a single model in an aggregate stress step 312. FIG. 5 illustrates the aggregate stresses on the joints of the skeletal doll 200 of one model 510. The model 510 represents the stresses associated with completion of one sequence of tasks. This modeling method is then applied forward to identify a full work routine of an individual user and aggregate the stresses associated with the day to day work routine in the aggregate stress step 312. In some examples, the process 300 can further aggregate day to day into weeks, weeks into months, etc.

The determined aggregate stresses are then compared to allowable stress limits in a comparison 314. In order to perform the comparison, the process identifies the medically recommended allowed repetitive bone joints movements for each type of movement included within the sequence(s). The aggregate model 510, determined at step 312, determines the aggregate stresses and loads on each joint 210 during the different activities forming a full sequence. This aggregate stress and load is compared to the identified medical limits (e.g., the load and stress limits that can be applied before an injury or repetitive stress damage is likely to occur). When the aggregate stresses and/or loads exceed the medical limits (or guidelines) for a given joint 210, the process instructs a workspace designer to redesign the tasks and/or redesign he workflow of the specific user to reduce the aggregate stresses in a redesign tasks step 316. When the aggregate stresses do not exceed the medical limits, the process 300 indicates that the workflow and/or workspace is approved, and the process 300 implements the task sequence in an implement task flow step 318.

By using the human skeletal doll 200, and modeling the actual tasks and task sequences within the simulation, the process 300 acts as a prophylaxis against repetitive stress injuries and/or damage, as well as against any similar damage.

In some implementations, the process 300 can be further expanded by incorporating an identify beneficial tasks step after aggregating the stresses. In this step, the model identifies any movements or operations that may beneficially improve musculoskeletal health by comparing the motions within the tasks to a bank of beneficial exercises. When tasks can be achieved via motions that match or approximate the beneficial exercises, instructions are generated and provided to the users performing the tasks identifying the procedure for beneficially performing the task. This can be performed live via an augmented reality system or provided to the users via instructions before the workflow begins.

While described above as being utilized with regards to a human joint and movement system, it is appreciated that the same techniques for analysis and workflow planning may be applied to robots including articulating parts in order to identify and reduce articulation related stresses on the machinery.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for providing a prophylaxis against injuries by identifying a set of tasks within a workflow comprising:
simulating performance of the tasks using a skeletal model within a computer simulation, the skeletal model being defined by joints and articulations of the joints;
aggregating stresses at the joints over time into an aggregate stress and an aggregate load;
comparing the stresses to a recommended stress limit and a recommended load limit; and
recommending a reconfiguration of the set of tasks in response to one of the stresses exceeding the recommended stress limits and the loads exceeding the recommended load limits.

2. The computer-implemented method of claim 1, wherein the skeletal model defines a corresponding degree of freedom and a type of movement of the joints.

3. The computer-implemented method of claim 2, wherein the type of movement includes flexion/extension, abduction, adduction, and rotation.

4. The computer-implemented method of claim 1, wherein the skeletal model is a human musculoskeletal model.

5. The computer-implemented method of claim 4, wherein the recommended stress limit and the recommended load limit are specific to a unique worker's height, weight, reach, and age.

6. The computer-implemented method of claim 5, wherein simulating performance of the tasks includes identifying one or more activities within a task and modeling joint movements of performance of the one or more activities using the skeletal model.

7. The computer-implemented method of claim 6, wherein simulating performance of the tasks includes simulating load variation and frequency variation of the joint movements using the skeletal model.

8. The computer-implemented method of claim 1, wherein comparing the stresses to a recommended stress limit and a recommended load limit further comprises comparing an articulation of one or more joint to a beneficial articulation and wherein recommending a reconfiguration of the set of tasks includes recommending modifying performance of the task to include the beneficial articulation.

9. The computer-implemented method of claim 8, wherein recommending modifying performance of the task to include the beneficial articulation includes displaying the modified performance to a user via an augmented reality device.

10. A computer program product comprising a non-transitory storage medium storing instructions for causing a computer system to implement a process for providing a prophylaxis against injuries by identifying a set of tasks within a workflow, the process comprising:
simulating performance of the tasks using a skeletal model within a computer simulation, the skeletal model being defined by joints and articulations of the joints;
aggregating stresses at the joints over time into an aggregate stress and an aggregate load;
comparing the stresses to a recommended stress limit and a recommended load limit; and
recommending a reconfiguration of the set of tasks in response to one of the stresses exceeding the recommended stress limits and the loads exceeding the recommended load limits.

11. The computer program product of claim 10, wherein the skeletal model defines a corresponding degree of freedom and a type of movement of the joints.

12. The computer program product of claim 11, wherein the type of movement includes flexion/extension, abduction, adduction, and rotation.

13. The computer program product of claim 10, wherein the skeletal model is a human musculoskeletal model.

14. The computer program product of claim 13, wherein the recommended stress limit and the recommended load limit are specific to a unique worker's height, weight, reach, and age.

15. The computer program product of claim 14, wherein simulating performance of the tasks includes identifying one or more activities within a task and modeling joint movements of performance of the one or more activities using the skeletal model.

16. The computer program product of claim 15, wherein simulating performance of the tasks includes simulating load variation and frequency variation of the joint movements using the skeletal model.

17. The computer program product of claim 10, wherein comparing the stresses to a recommended stress limit and a recommended load limit further comprises comparing an articulation of one or more joint to a beneficial articulation and wherein recommending a reconfiguration of the set of tasks includes recommending modifying performance of the task to include the beneficial articulation.

18. The computer program product of claim 17, wherein recommending modifying performance of the task to include the beneficial articulation includes displaying the modified performance to a user via an augmented reality device.

19. A system comprising:
a computer having a set of processing circuitry and a persistent storage, the persistent storage being configured to cause the set of processing circuitry to operate a method including for providing a prophylaxis against injuries by identifying a set of tasks within a workflow, the process comprising:
simulating performance of the tasks using a skeletal model within a computer simulation, the skeletal model being defined by joints and articulations of the joints;
aggregating stresses at the joints over time into an aggregate stress and an aggregate load;
comparing the stresses to a recommended stress limit and a recommended load limit; and
recommending a reconfiguration of the set of tasks in response to one of the stresses exceeding the recommended stress limits and the loads exceeding the recommended load limits.

20. The system of claim 19, wherein the computer is in communication with a plurality of sensors distributed about a work environment via an internet of things (IoT) configuration, and wherein identifying the set of tasks within the workflow includes analyzing an output of the plurality of sensors.
